(19) United States
(12) Patent Application Publication
(10) Pub. No.: US 2020/0297388 A1
(43) Pub. Date: Sep. 24, 2020

(54) SYSTEM, COMPONENTS AND METHOD FOR TREATING MAXILLARY DEFICIENCIES AND CRANIOFACIAL DYSTROPHY

(71) Applicant: Cameron Kaveh, Bell Canyon, CA (US)

(72) Inventor: Cameron Kaveh, Bell Canyon, CA (US)

(21) Appl. No.: 16/630,818

(22) PCT Filed: Jul. 15, 2018
(86) PCT No.: PCT/US2018/042200

§ 371 Date: Jan. 13, 2020

Related U.S. Application Data
(60) Provisional application No. 62/668,812, filed on May 8, 2018.
(60) Provisional application No. 62/533,010, filed on Jul. 15, 2017.

(51) Int. Cl. A61B 17/66

(52) U.S. Cl. CPC A61B 17/663

(57) ABSTRACT

A system, components and methods is described to enable the treatment of maxillary deficiency and craniofacial dystrophy by positioning the maxilla via protraction forces applied extra-orally to skeletal anchorage devices embodied as bone anchors. The protraction forces are applied with minimal or no rotational moments being applied at the bone anchors. The shape and geometry of the bone anchors can be customized to fit the particular skeletal of individual patients.

### RELATED APPLICATIONS

The present invention claims priority to U.S. Provisional Application 62/533,010, filed Jul. 15, 2017; and further claims priority to U.S. Provisional Application 62/668,812, filed May 8, 2018; both of which are incorporated herein by reference.

### FIELD OF THE INVENTION

The present invention is directed to the field of orthodontics and the use of skeletal anchorage devices. The present invention is also directed to a system, components and methods that enable forward advancement and growth of the maxilla and other skeletal bones coupled to the maxilla. The present invention in further directed to skeletal anchorage devices for treating maxillary deficiency and craniofacial dystrophy.

### BACKGROUND

Traditional orthodontics focuses primarily on straightening misaligned teeth. The main goal is to create a great smile with perfect tooth alignment and proper bite. Braces and wires are the preferred way of aligning the teeth.

Other areas of orthodontics are directed to achieving good jaw alignment in children. These areas encompass modification and movement of the bones that support the teeth to attain desirable changes in their relative position so that aesthetics, function, and oral health are improved. Treatments are centered on bone movement rather than tooth movement and address the underlying causes of bad bites and misaligned teeth by utilizing fixed or removable dental appliances, as opposed to braces. Tooth alignment may be done later, if desired, with braces or with non-brace aligners. If begun at an early age, modification and movement of bones can obviate the need for the extraction of adult teeth or jaw surgery, and minimize or completely eliminate the need for fixed braces during adolescence. Because the bone structures of adults are fully formed, dental appliances are not well suited for jaw alignment in adults.

Craniofacial dystrophy and maxillary hypoplasia are a type of malocclusion, which is a facial growth pattern characterized by deficient jaw growth that results in excessive vertical and lack of horizontal growth of the jaws and that that give the appearance of a long face with a weak chin. Treatments for craniofacial dystrophy include plastic surgery and movement of the teeth. Detractors of these treatments suggest that it merely masks the craniofacial dystrophy without addressing the underlying improperly formed facial bone structure. Other treatments rely on very invasive and complicated intra-oral prone surgeries that require cutting and grafting of bones.

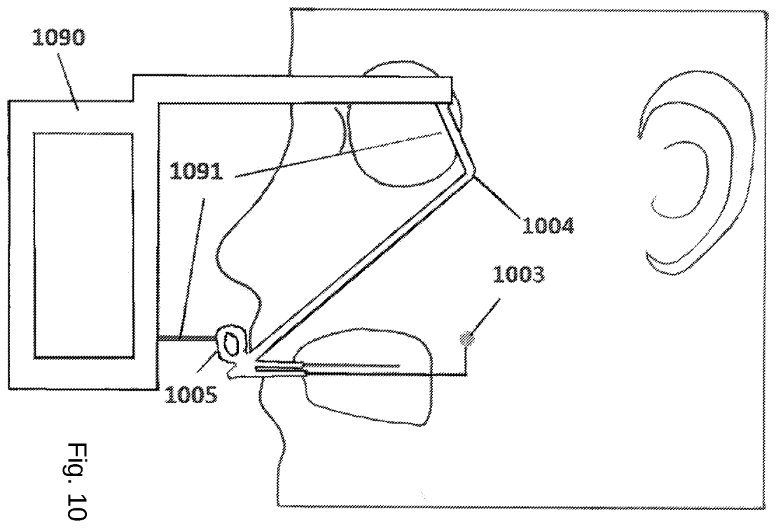

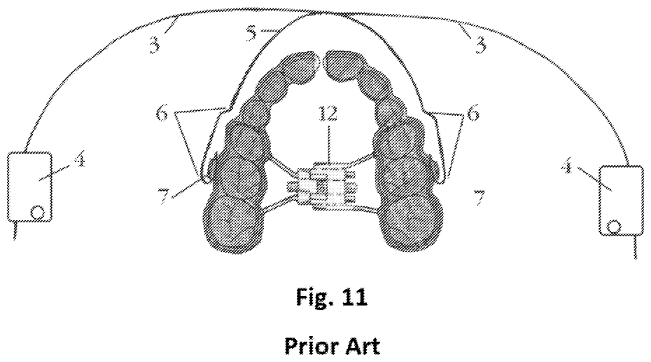

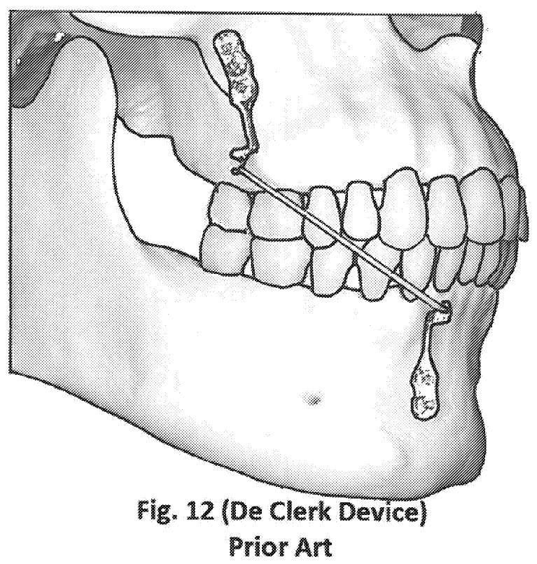

Recently, fixed and removable appliances, for example, Biobloc, acrylic expander, facemask, Bollards, have been developed for orthotropic treatment of Class III adult malocclusion. For example, a device known as the Keles Facemask (see FIG. 11) includes a palatal expander and an orthodontic face bow, both of which attach to molar bands that are fixed to a patient's dentition. The application of external forces via the face bow to the molars is used to create forward movement and growth of the maxilla. However, the jaw movement imparted by the Keles device includes a rotational component, which also causes forwardly directed downward growth of the maxilla. It has been identified that the Keles device and similar devices are not the best solution for treatment of craniofacial dystrophy since this form of malocclusion is best treated via non-rotational forward movement and growth to the maxilla and the 9 bones that articulate with the maxilla. The Keles device also relies on tooth borne forces to achieve its movement, which is also less than ideal, since movement that might otherwise be imparted to the maxilla bone is instead imparted to teeth. Another device invented by De Clerck (see FIG. 12) is a Bollard miniplate bone anchor that is used to transfer intra-orally generated forces to the maxilla. The Bollard device as well causes forwardly directed downward movement and growth of the maxilla. The Bollard miniplate bone anchor also utilizes a “one size fits all” approach that does not accurately take into account patient specific features such as bone thickness, craniofacial symmetry, and bone surface area/geometry, which eliminates the ability to optimally place and clinically use the device. For example, the Bollard device has a standardized neck length. This standardized length limits its use, where when one end of the Bollard device is attached in the keratinized tissue at or below the mucogingival junction, the location of the opposite end cannot be optimized to account for the particular different skeletal geometries of different patients. Moreover, current marketed devices are all manufactured as a relatively flat surface at their plate end, requiring surgeon to manually manipulate the device to fit to a patient's bone geometry. This approach is flawed in that manually manipulating the miniplate is dependent on clinician's physical skill. Oftentimes, the miniplate needs to be manipulated mid-surgery, increasing procedural time and opportunities for adverse events to occur. Additionally, requiring manual manipulation of the bone anchor requires a sacrifice in material properties. That is, stiffer, more strong materials cannot be used because of their inability to be manipulated to fit individual patient's bone geometry. Lastly, following manual manipulation of the Bollard device, its material properties can be greatly degraded. As a result, fracture risk and risk of performance failure greatly increases. In fact, the instructions for use of the Bollard miniplate specifically state “bending should be limited to the region between the holes (1) in the miniplate. This bending should not exceed 10° and may only be performed once. Bending of the plate more than 10° and/or repetitive bending may lead to fracture during or after surgery. The angulation between the miniplate and the neck (2) should not be modified in order to ensure good contact between the lower part of the neck and the alveolar bone (3). The round bar should not be bended. Bending of the round bar may lead to fracture during or after surgery.” All these limitations do not allow for optimization of the Bollard device installation location, and therefore, limit its potential effective use.

To date, no known systems, components or methods that use protraction force are able to optimally treat craniofacial dystrophy and maxillary hypoplasia in adults, as well as children, without introducing rotation of the maxilla.

### SUMMARY OF INVENTION

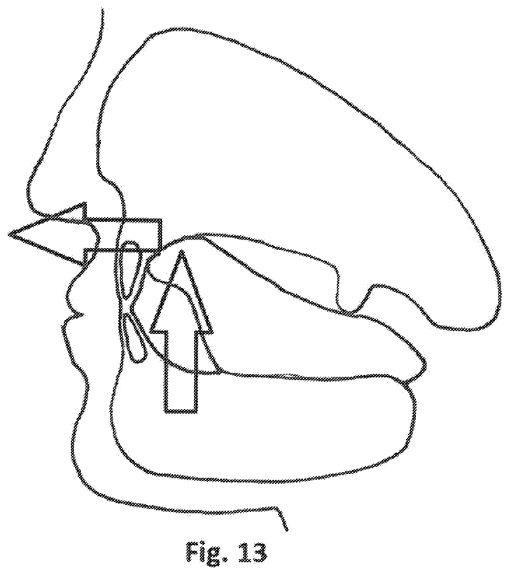

The present invention identifies that in the mouths of normally developed individuals, vector forces applied to the palate by the tongue (see direction of arrows in FIG. 13) causes normal facial and skeletal growth. The present invention further identifies that in subject's whose skeletal growth is deficient and whose tongue is unable to provide sufficient forces against the palate to effectuate such growth, such as in adults with craniofacial dystrophy, forward and, as may be needed, additional upward facial growth can be engineered via application of extra-orally generated forward or forward and upward directed protraction forces to skeletal anchorage devices (also referred to as bone anchors below) coupled to the maxilla.

Unlike known solutions, the present invention also identifies that individual patients can be provided more optimal clinical outcomes when the shape of their skeletal anchorage devices is customized to their individual and unique bone structure.

Before providing a customized skeletal anchorage device, the present invention utilizes software analysis of patient specific 3D data/model derived from cone beam computed tomography (CBCT), CAT Scan, or magnetic resonance imaging (MRI). Each patient's bone thickness and geometry is assessed to optimize where one end of the bone anchor is to be attached via bone screw fixation as well as to optimize the geometry of a mounting plate at the end so that it beneficially exits, keratinized tissue in a manner that minimizes infection. The 3D data/model is also used to customize the geometry of each skeletal anchorage device to match each patient's unique attributes such that it is optimally placed within the patient's mouth to minimize chafing and rubbing as well as to correct placement of its second end so that force(s) applied to the second end do not cause excessive moments at the point to the skull at the other end.

Taken together, this novel approach generates much greater predictability and accuracy in deriving skeletal anchorage, which results in overall greater patient outcomes.

The present invention's patient-specific approach enables a plate of each skeletal anchorage device to be attached flush against the patient's bone, and as well allows design a neck of each device with a geometry that enables intraoral neck placement against contours of the jaw.

The present invention also enables a customized screw configuration based on the patient's bone geometry, density and thickness. Screw positions in a connecting plate can be optimized to take advantage of skull locations with optimal bone density and thickness, and a force transmitting neck can be designed to attach to the screws in a manner that optimizes force distribution in the screw, thereby reducing the stress concentrations on the screws, enabling more effective and capable orthodontic treatment and procedures. This approach promotes better osseointegration, minimizes complications, involves greater predictability for clinicians, and overall improves performance and patient comfort.

While the present invention is directed to positioning the maxilla via protraction forces to treat craniofacial dystrophy and maxillary hypoplasia, the scope of the invention anticipates its possible use as a patient-specific mandibular anchor for mandibular repositioning, namely, encouraging mandibular forward positioning. Accordingly, the present invention contemplates that it can also more broadly be used for dentofacial orthopedics.

In one embodiment, the present invention comprises a system to treat maxillary deficiency, the system comprising: an orthodontic face bow comprising an intra-oral portion; and an extra-oral portion, wherein the extra-oral portion is configured to receive one or more extra-oral protraction force, and wherein the intra-oral portion comprises one or more coupler configured to transfer the one or more extra-oral protraction force to intra-oral portions of the patient's mouth that are not teeth of the patient. In one embodiment, the face bow consists of the one or more coupler. In one embodiment, the one or more coupler comprises a bone anchor. In one embodiment, the intra-oral portions of the patient's mouth that are not teeth comprise maxilla of the patient. In one embodiment, the intra-oral end comprises silicone, plastic, acrylic, polymer, or combination thereof. In one embodiment, the intra-oral portions of the patient's mouth that are not teeth comprise a maxilla. In one embodiment, the present invention comprises one or more protraction device configured to apply the one or more extra-oral force.

In one embodiment, the present invention comprises a system for treating a maxillary deficiency of a subject comprised of a first face bow comprised of an intra oral-end; and an extra-oral end, wherein the extra-oral end is configured to receive a first force, and wherein the intra-oral end is configured to transfer the first force to a maxilla of the subject; and a second face bow, comprised of an intra oral-end; and an extra-oral end, wherein the extra-oral end is configured to receive a second force, and wherein the intra-oral end is configured to transfer the second force to the subject's maxilla In one embodiment, the invention comprises an external protraction device, wherein the external protraction device is configured to apply the first force to the extra-oral end of the first face bow and to apply the second force to the extra-oral end of the second face bow.

In one embodiment, the present invention comprises: a kit for treating a maxillary deficiency of a subject, the kit comprising: a container; at least one bone anchor, wherein the container is configured to store the at least one bone anchor, wherein the at least one bone anchor is configured to transfer extra-oral forces to non-dental portions of the patient's mouth. In one embodiment the kit further comprises at least one coupler, the at least coupler comprised of a first end and a second end, wherein the first end is configured to be coupled to a first face bow, and wherein the second end is configured to be coupled to the bone anchor. In one embodiment the kit further comprises a second face bow configured to transfer extra-oral forces directly to the subject's maxillary tuberosity or superior palate. In one embodiment the kit further comprises comprising at least one screw type fastener configured to attach the at least one bone anchor to the non-dental portions of the patient's mouth. In one embodiment, the at least one bone anchor comprises grade 4 or grade 5 titanium. In one embodiment, the at least one bone anchor is a printed from metal.

In one embodiment, the present invention comprises: a bone anchor for transferring an extra-oral force to a subject's skull, comprising: a first end and a second end, wherein the second end is configured to be intra-orally coupled to the skull, wherein the first end comprises a first coupler, and where the first coupler is configured to be coupled to a force generated from outside the subject's mouth. In one embodiment the bone anchor comprises comprising a connecting piece defined by a length disposed between the first end and the second end. In one embodiment, the second end comprises a plate within which a plurality of apertures are disposed. In one embodiment, the plate comprises non-planar surfaces configured to fit against non-planar surfaces of the subject's skull. In one embodiment, the plurality of apertures consists of four apertures. In one embodiment, the plurality of apertures consist of at least four apertures wherein, relative to an axis defined by or extending from the length, an equal number of the apertures are disposed on opposite sides of the axis. In one embodiment, the first coupler comprises an aperture or protrusion configured to be coupled to a second coupler or to an end of a face bow. In one embodiment, the bone anchor comprises Titanium 4 or Titanium 5. In one embodiment, the bone anchor is a printed from metal. In one embodiment, when viewed in a cross-section, the connecting piece comprises one or more of a flat, rounded, and a curved surface. In one embodiment, the first coupler comprises a cylinder.

In one embodiment, the present invention comprises a system for transferring an extra-oral force to a maxilla of a subject, the system comprising: a first part and a second part, wherein the second part is coupled to the first part, wherein the first part is configured to receive the extra-oral force and transfer the extra-oral force to the second part, and wherein the second part is configured to transfer the force to the subject's maxilla. In one embodiment, the first part comprises a coupler. In one embodiment, the second part comprises a bone anchor.

In one embodiment, the present invention comprises a third part, wherein the first part is coupled to the third part between the first part and the second part, and wherein the third part is configured to receive the extra-oral force from outside the subject's body and to transfer the extra-oral force to the second part. In one embodiment, the third part comprises a face bow, wherein the face bow comprises a first end configured to be coupled to the coupler. In one embodiment, the bone anchor comprises Titanium 4 or Titanium 5. In one embodiment, the bone anchor is a printed from metal. In one embodiment, the first part and the second part are connected by a connecting piece, wherein when viewed in a cross-section, the connecting piece comprises one or more of a flat, rounded, and a curved surface.

In one embodiment, the present invention comprises a method of treating maxillary deficiencies, comprising the steps of: intra-orally attaching at least two bone anchors to locations on the maxilla of a subject; coupling first ends of a face bow to the two bone anchors; and applying extra-oral force to the face bow. In one embodiment, the method comprises applying the extra-oral force that does not cause rotation of the maxilla about the at least two bone anchors. In one embodiment, with the subject standing and with the head of the subject facing forward, the extra-oral force is applied to the face bow only in a forward of a combination of forward and upward direction. In one embodiment, the bone anchor is not attached to any other intra-oral structure within the subject's mouth. In one embodiment, application of the extra-oral forces does not cause rotational moments to be generated at the two bone anchors. In one embodiment, the extra-oral force is applied to the face bow along a vector that passes through the face bow where the force is applied and the two bone anchors.

In one embodiment, the present invention comprises a method of treating a subject, comprising the steps of: intra-orally coupling a first end of face bow to the maxilla of the subject; and applying an extra-oral force to a second end of the face bow to cause the maxilla of the subject to move substantially forward without any downward rotation. In one embodiment, the first end of the face bow is coupled to the zygomatic buttress or the infrazygomatic crest of the subject. In one embodiment, the face bow is not attached to any of the teeth of the subject.

In one embodiment, the present invention comprises a method of causing a maxilla to grow via an application of a force, comprising the steps of: intra-orally coupling at least one bone anchor to the maxilla; generating an extra oral force; and coupling the extra-oral force to the at least one bone anchor to cause the maxilla to move forward without any downward rotation. In one embodiment, the force is coupled to the at least one bone anchor via a face bow. In one embodiment, the at least one bone anchor comprises two bones anchors, wherein each respective bone anchor is coupled to the maxilla on a respective opposite side of the maxilla. In one embodiment no other forces other than the extra-oral force are coupled to the at least one bone anchor.

In one embodiment, the present invention comprises a method for treating a subject, comprising the steps of: generating an extra-oral force; attaching at least one bone anchor to a maxilla of the subject; and coupling the extra-oral force to the at least one bone anchor to cause movement of maxilla and without causing moments to be generated at the at least one bone anchor.

In one embodiment, the present invention comprises A method of treating a subject, comprising the steps of: generating an extra-oral force; attaching at least bone anchor to a maxilla of the subject; and coupling the extra-oral force to the at least one bone anchor to non-rotationally move the maxillary complex of the subject about the at least one bone anchor.

In one embodiment, the present invention comprises a method of treating a subject for cranial dystrophy and deficiency comprising the steps of: generating an extra-oral force; attaching at least one bone anchor to a maxilla of the subject; and coupling the extra-oral force to the at least bone anchor to cause the maxillary complex of the subject to move in a direction that is not directed downward relative to a forward facing direction of the subject's face.

In one embodiment, the present invention comprises a method of treating a subject, comprising the steps of: attaching at least one skeletal anchorage device to a maxilla of the subject at an attachment point; and applying an extra oral force to the skeletal anchorage device, where the extra-oral force creates substantially no moment about the attachment point.

In one embodiment, the present invention comprises at least one face bow, comprising intra oral-ends; and at least one pair of extra-oral ends coupled to the intra-oral ends, wherein the extra-oral ends are configured to receive extra-oral forces, and wherein the intra-oral ends are configured to transfer the extra-oral forces to the interior of a subject's mouth without contacting the subject's teeth. In one embodiment, the at least one face bow comprises two face bows. In one embodiment, the intra-oral ends are configured to transfer the extra-oral forces to the maxilla of the subject. In one embodiment, the intra-oral ends are configured to transfer the forces to a palate of the subject.

In one embodiment, the present invention comprises a bone anchorage device for the attachment of orthodontic appliances, comprising: a first end and a second end, wherein the second end is configured to be directly coupled to an intra-oral location on the jaw of a subject, wherein the first end comprises a first coupler configured to be coupled to an orthodontic appliance inside or outside the subject's mouth; and a connecting piece with a length connecting the first end and the second end, wherein the length is comprised of one or more of a straight, bent, curved, and/or twisted portion. In one embodiment, the length is defined by one or more cross-sectional shape comprised of at least one rounded, elliptical, semicircular, curved or flat side. In one embodiment, the one or more cross-sectional shape comprises two or more of a rounded, elliptical, semicircular, curved or flat side. In one embodiment, the second end comprises a plate within which a plurality of apertures are disposed. In one embodiment, the plate comprises a surface, wherein the surface is non-planar; and wherein a substantial portion of the non-planar surface is configured to conform to a substantial portion of a surface of the jaw. In one embodiment, the first coupler comprises an attachment point. In one embodiment, the attachment point comprises an aperture. In one embodiment, the plate comprises a left and right portion, wherein the left and right portion each comprise at least two apertures, wherein the apertures in the left portion are disposed along an axis that is generally slanted with respect to an axis of the connecting piece at its connection to the plate, and wherein the apertures in the right portion are disposed along an axis that is generally parallel with respect to the axis of the connecting piece at its connection to the plate. In one embodiment, the plate comprises a left and right portion, wherein the left and right portion each comprise at least two apertures, wherein the apertures in the right portion are disposed along an axis that is generally slanted with respect to an axis of the connecting piece at its connection to the plate, and wherein the apertures in the left portion are disposed along an axis that is generally parallel with respect to the axis of the connecting piece at its connection to the plate. In one embodiment, the plate comprises a left and right portion, wherein the left and right portion each comprise two apertures, and wherein the apertures are disposed asymmetrically with respect to each other. In one embodiment, the plate comprises a left and right portion, wherein the left and right portion each comprise two apertures, and wherein the apertures are disposed symmetrically about an axis defined by the connecting piece. In one embodiment, the location on the jaw comprises a location on a zygomaticomaxillary buttress or the mandible. In one embodiment, the location on the jaw comprises a location on a nasomaxillary buttress or the mandible. In one embodiment, the location on the jaw comprises a location on a maxillary buttress or the mandible. In one embodiment, the location on the jaw comprises a location on the maxilla or mandible. In one embodiment, the location on the jaw is adjacent a zygomatic suture on the maxilla or on the mandible. In one embodiment, the first coupler comprises an attachment point configured to permit attachment of an orthodontic appliance. In one embodiment, the orthodontic appliance comprises a face bow. In one embodiment, the orthodontic appliance comprises a second coupler.

In one embodiment the present invention comprise a method of forming a patient-specific bone anchorage device, comprising the steps of: obtaining a model of a patient's skull or mandible, identifying one or more location on the model of the patient's skull or mandible; manipulating the model of the bone anchorage device to cause a shape of the bone anchorage device to fit against the one or more location on the model of the patient's skull or mandible; and positioning the model of a bone anchorage device against the model of the patient's skull or mandible. In one embodiment, the model of the patient's skull is a digital model obtained with a digital scanning device, wherein the model of the bone anchorage device is a digital model embodied in code or memory of a computing device; and wherein manipulating the shape of the model of the bone anchorage device is performed on the computing device. In one embodiment, the digital model of the patient's skull represents a surface of the skull or mandible. In one embodiment, the digital model of the patient's skull or mandible, and the digital model of the bone anchorage device are displayed on a digital display. In one embodiment, the manipulation of the bone anchorage device comprises lengthening, shortening, contouring, twisting, stretching, and/or bending a connecting piece of the bone anchorage device. In one embodiment, the manipulation of the bone anchorage device comprises changing a contour of a plate at the second top end of the bone anchorage device. In one embodiment, the manipulation of the bone anchorage device comprises changing a thickness of the bone anchorage device. In one embodiment, the manipulation of the bone anchorage device comprises manipulating a first bottom end of the bone anchorage device. In one embodiment, the digital model of the bone anchorage device is stored in a file In one embodiment, the file comprises an STL file. In one embodiment, comprises manufacturing the bone anchorage device based on digital data stored in the file. In one embodiment, manufacturing the bone anchorage device is performed by printing. In one embodiment, the bone anchorage device comprises metal. In one embodiment, bone anchorage device comprises titanium 5. In one embodiment, the manipulation comprises twisting, stretching, and or bending one or more portion of the model of the bone anchorage device. In one embodiment, the one or more location comprises a location on a zygomaticomaxillary buttress. In one embodiment, the one or more location comprises a location on a nasomaxillary buttress. In one embodiment, the one or more location comprises a location on a maxillary buttress. In one embodiment, the one or more location is adjacent a zygomatic suture on the maxilla. In one embodiment, further comprises a step of using screws to attach the bone anchorage device to a skull or mandible of the patient. In one embodiment, the bone anchorage device comprises an attachment plate comprised of screw holes, wherein the manipulation includes locating the screw holes in the attachment plate such that when mounted to the patient's skull or mandible by screws inserted within the screw holes and with an external force applied to the bone anchorage device, the external force is distributed to be substantially equal among the screws. In one embodiment, the one or more location comprises a location on the skull mandible that optimizes the force distribution. In one embodiment, the one or more location comprises a location on the e that has a bone thickness and/or density capable of optimizing the force distribution. In one embodiment, the one or more location is identified by a person performing the method. In one embodiment, the one or more location is identified using artificial intelligence.

In one embodiment, the present invention comprises: a method of using a patient-specific bone anchorage device, comprising the steps of: identifying one or more location adjacent to a zygomatic suture of a patient; manipulating a shape of the bone anchorage device to cause the shape to fit against the patient's skull in the area of the zygomatic suture; attaching the bone anchorage device to the one or more location. In one embodiment, the present invention further comprises applying an extra-oral force to the bone anchorage device. In one embodiment, the present invention comprises applying the extra-oral force to the bone anchorage device with little or no rotational moment created at the bone anchorage device by the extra-oral force.

Other embodiments, aspects, and benefits of the present invention will thus become apparent upon a further reading of the detailed description below.

### FIGURES

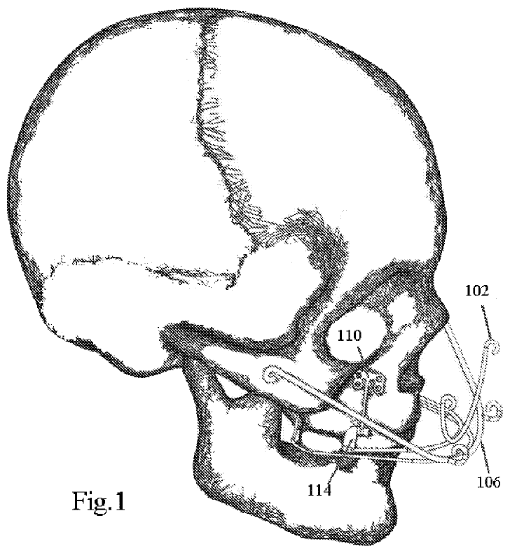

Referring to FIG. 1, there is seen a representation of a system used for engineering facial and skeletal growth.

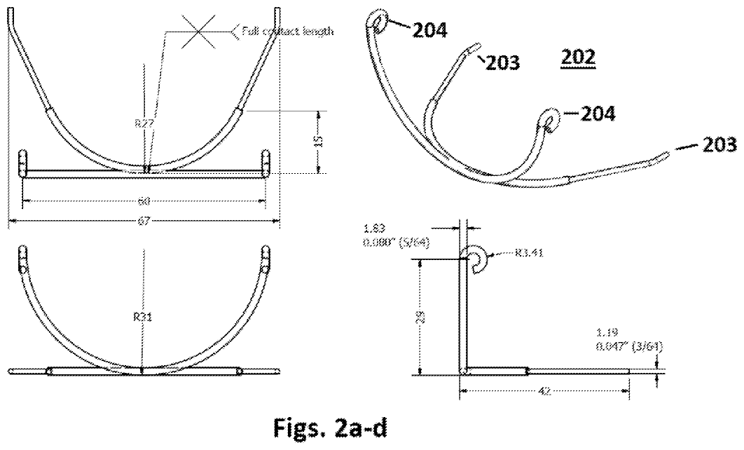

Referring to FIGS. 2a-d, there are seen top, front, side and perspective representations of an embodiment of a first face bow and a second face bow.

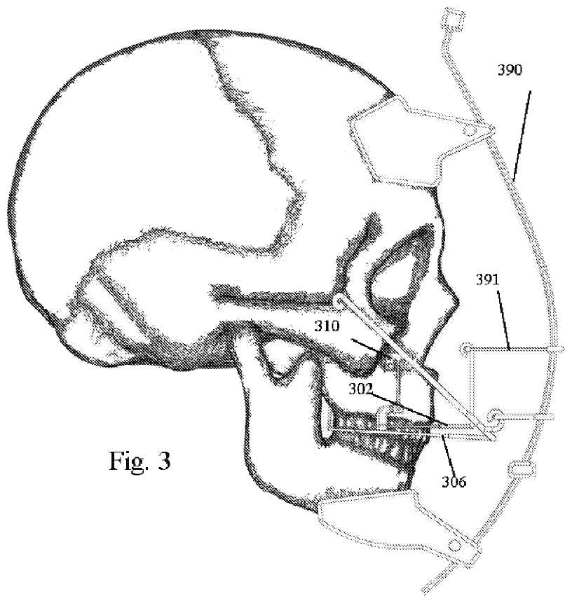

Referring to FIG. 3, there is seen a representation of an embodiment of a first face bow coupled to orthodontic headgear.

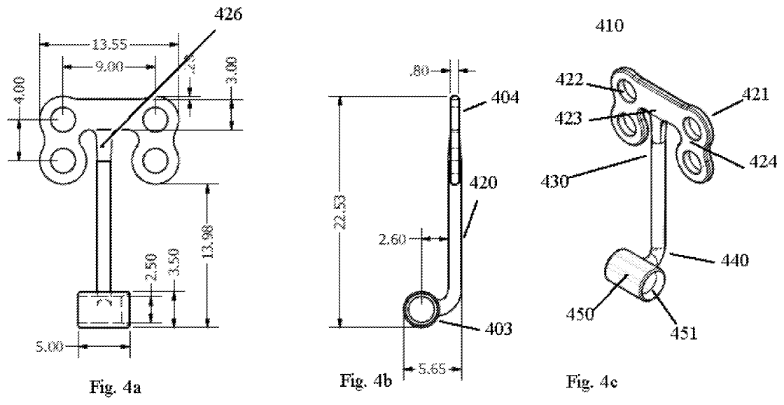

Referring to FIGS. 4a-c, there are seen top, front, side and perspective representations of an embodiment of a bone anchor.

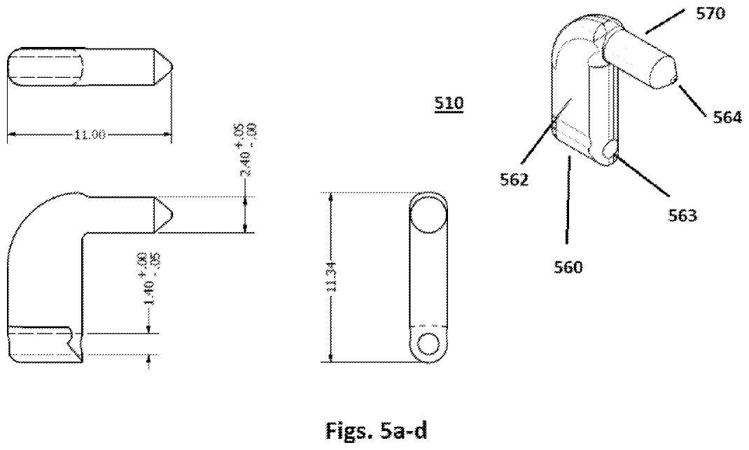

Referring to FIG. 5, there are seen top, a front, and perspective representations of an embodiment of a coupler.

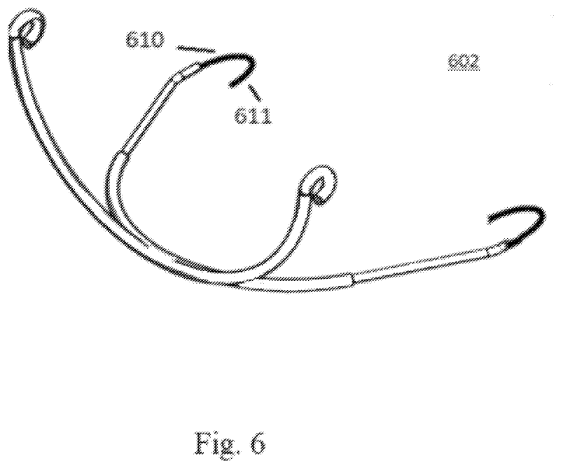

With reference to FIG. 6, there is seen another embodiment of a first face bow.

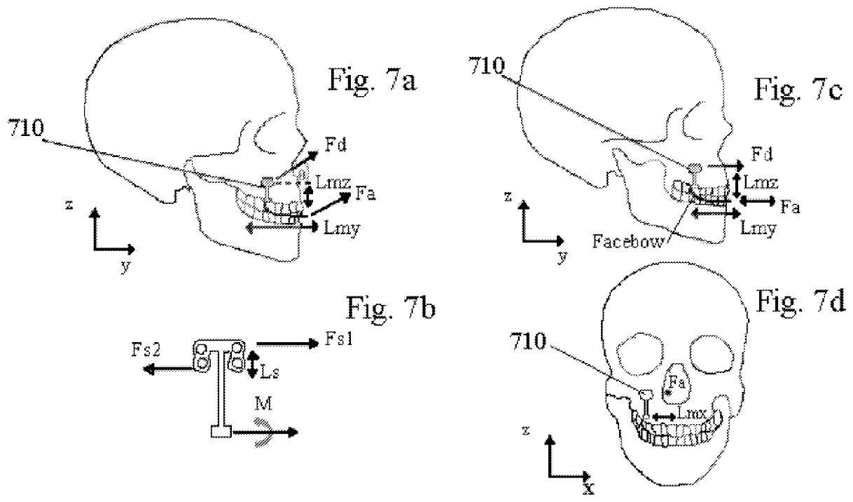

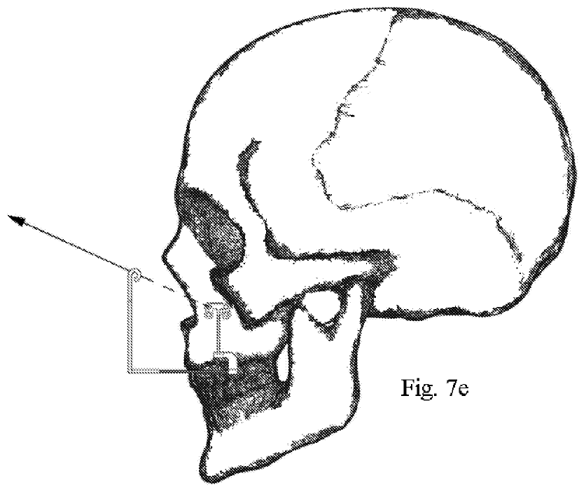

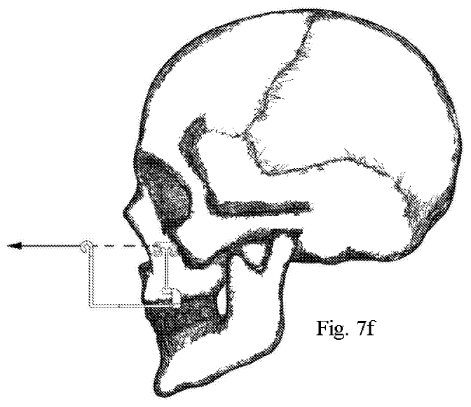

With reference to the representations in FIGS. 7a-f, methods of use of a first face bow and bone anchor are referenced.

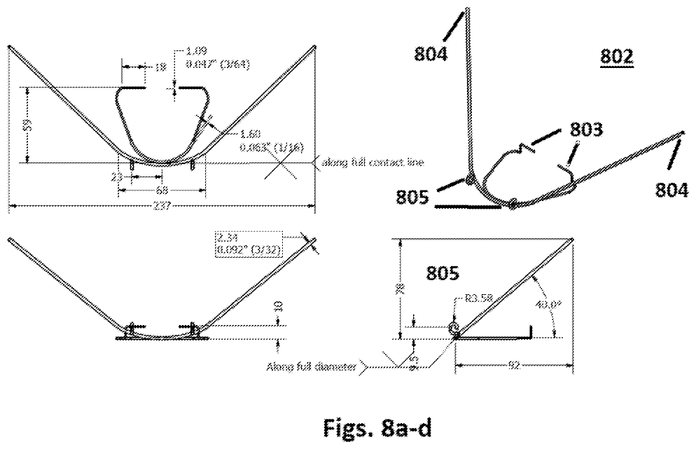

With reference to FIGS. 8a-d, there are seen representations of top, front, side, and perspective and views of an embodiment of the second face bow shown partially in FIG. 3 and more fully in FIG. 1.

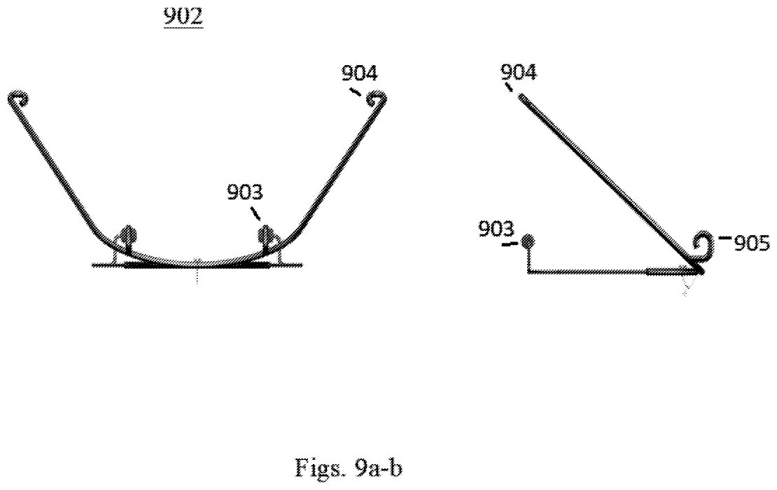

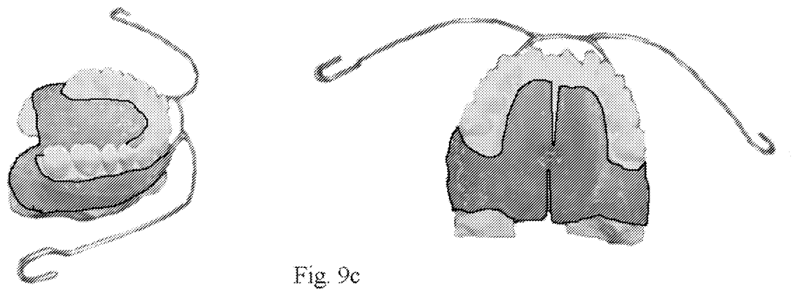

Referring to FIGS. 9a-b, there are seen representations of top and side views of another embodiment a second face bow.

Referring to FIG. 10, there is seen a representation of application of horizontal and upward forces by a second face bow to a maxilla of a subject.

Referring to FIG. 11, there is seen a prior art device.

Referring to FIG. 12, there is seen a prior art device.

Referring to FIG. 13, there are seen a representations of a bone screw.

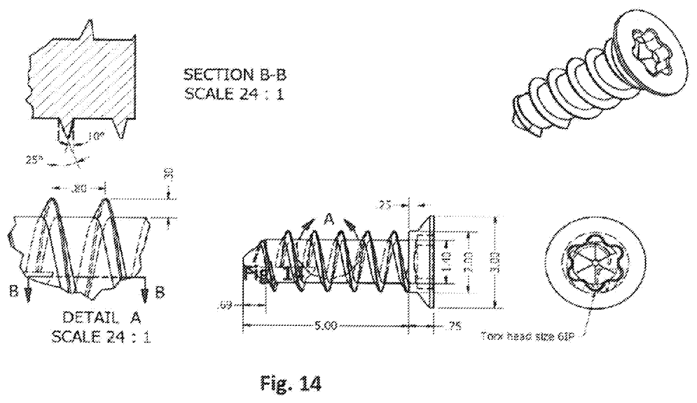

Referring to FIG. 14, there is seen a representation of forces applied to the palate by the tongue.

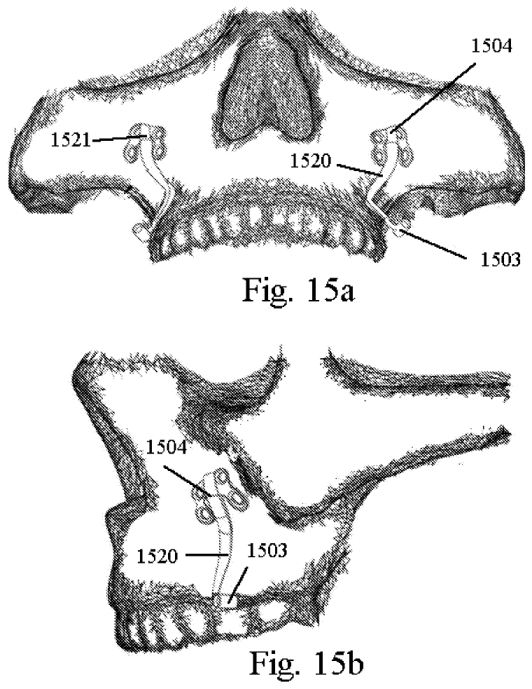

Referring to FIGS. 15a-b, there are seen representations of embodiments of a bone anchor.

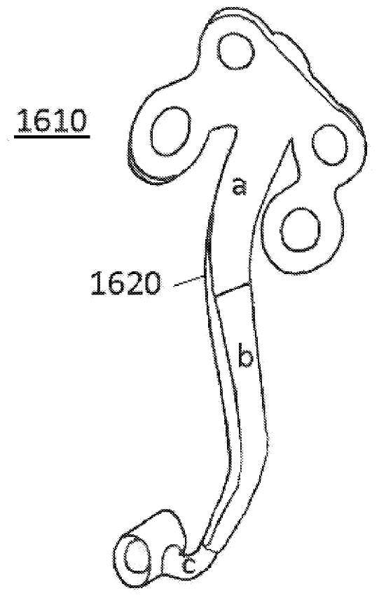

Referring to FIG. 16, there is seen another representation of a bone anchor embodiment.

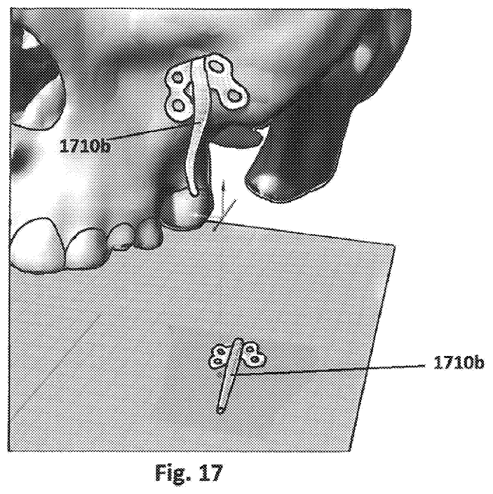

Referring to FIG. 17, there are seen virtual representations of a bone anchor.

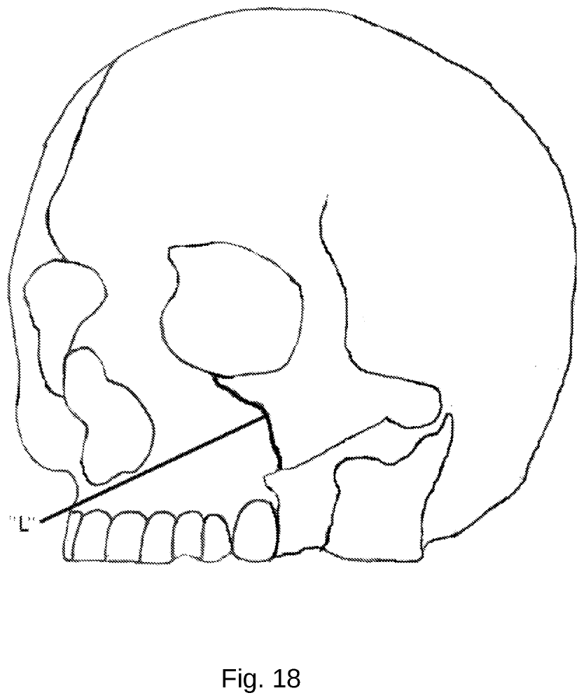

Referring to FIG. 18, there is seen a representation of the location of a zygomatic maxillary suture.

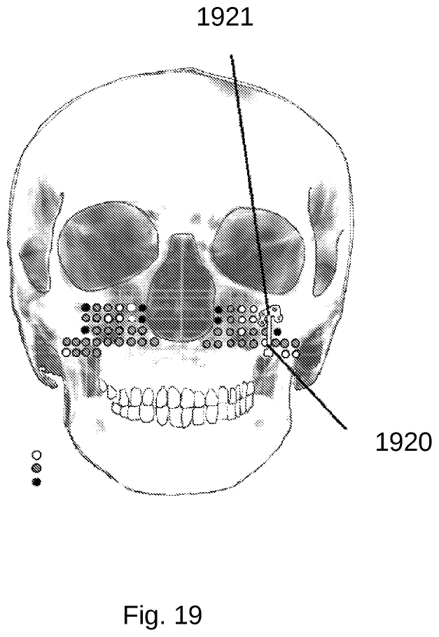

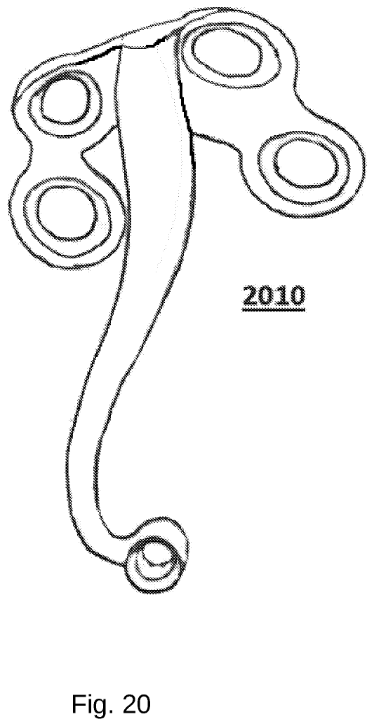

Referring to FIG. 19, there is seen a representation of a human skull.

Referring to FIG. 20, there is seen a representation of an embodiment of a bone anchor.

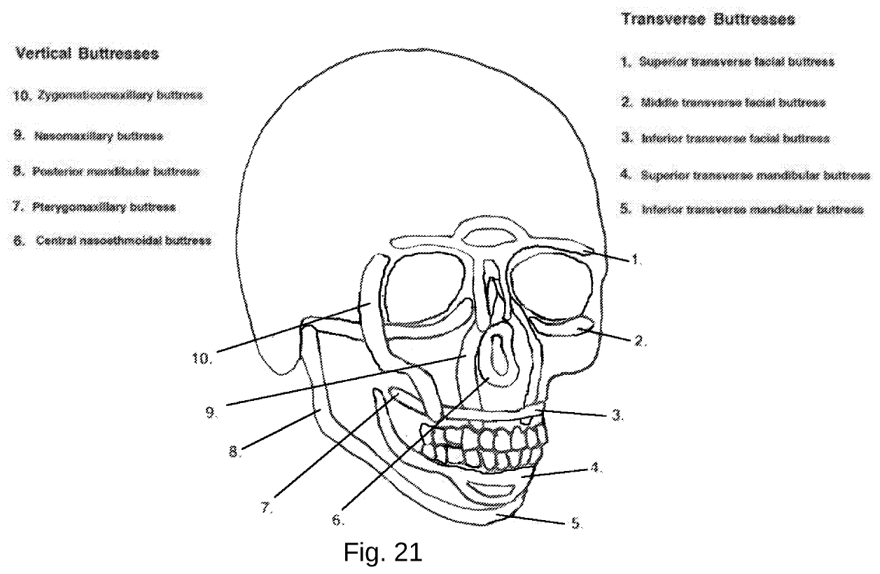

With reference to FIG. 21, there is seen a representation of the anatomy of a skull.

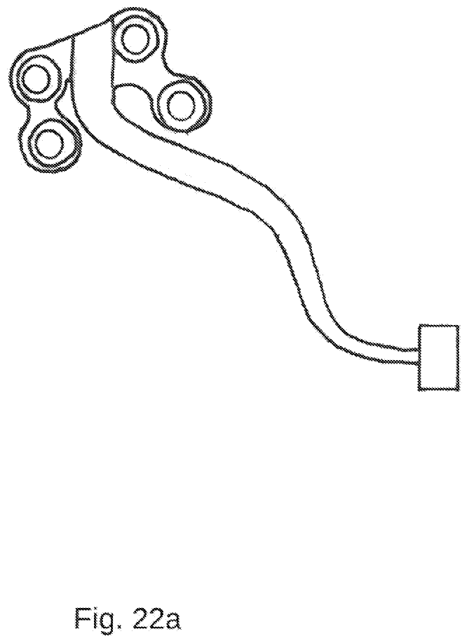

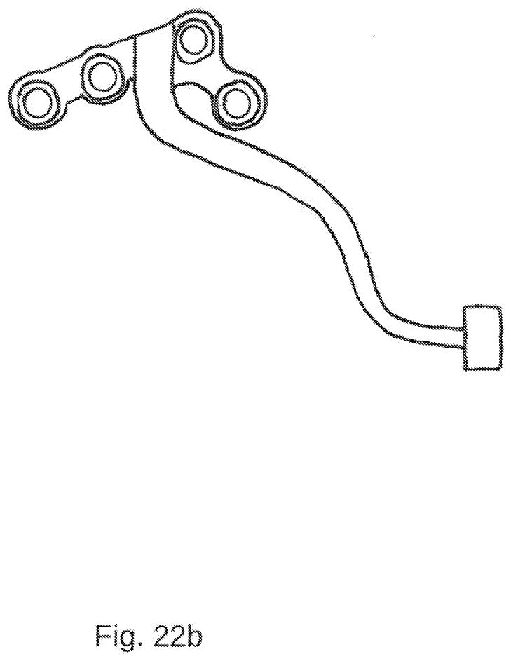

With reference to FIGS. 22a-b, there are seen embodiments of customized bone anchors.

### DETAILED DESCRIPTION

Referring to FIG. 1, there is seen a representation of a system used for engineering facial and skeletal growth as is needed to treat maxillary deficiencies, craniofacial dystrophy and maxillary hypoplasia via direct application and transfer of extra-oral protraction forces to a maxilla of a subject. In one embodiment, the system comprises: first face bow 102, second face bow 106, two bone anchors 110 (one which is not visible on the opposite side of the skull in FIG. 1), and two couplers 114 (one which is not visible on the opposite side of the skull in FIG. 1). As will be appreciated upon a reading of the descriptions provided below, to achieve beneficial facial growth of the maxilla, the first face bow 102 can be used alone, the second face bow 102 can be used alone, or both the first and second face bow can be used in combination.

Referring to FIGS. 2a-d, there are seen top, front, side and perspective representations of an embodiment of a first face bow and a second face bow. In one embodiment, a first face bow 202 comprises first ends 203 configured to be inserted into the oral cavity of a subject and second ends 204 configured to be coupled extra-orally to headgear (see FIG. 3) worn by a subject. The embodiment in FIG. 2 represents the second ends 204 being comprised of a loop, however, other geometries for the second ends are considered to be within the scope of the invention as long as such geometries enable the second ends to be coupled to an extra-oral headgear or other external structure capable of applying extra-oral forces to the second ends. In one embodiment, the first face bow 202 comprises 304V stainless steel and the exemplary dimensions shown in FIG. 2, however, other materials, geometries and other dimensions are within the scope of the invention as long as they are compatible for human use and are configured to transfer extra-oral protraction forces applied at the first ends 203 to the second ends 204 in the manner described further below.

Referring to FIG. 3, there is seen a representation of an embodiment of a first face bow coupled to orthodontic headgear. In one embodiment, first a first face bow 302 is coupled to a headgear 390 that acts as an anchor to for extra-oral protraction forces that are applied to the first face bow, which in turn transfers the extra-oral protraction forces to intra-orally mounted bone anchor 310. In one embodiment, headgear 390 is used in conjunction with a plurality of elastics 391 that generate the extra-oral protraction forces applied to the face bows. In other embodiments, it is contemplated that protraction forces can be provided by springs, wires or other means capable of applying tension forces to the face bows. In embodiments, the headgear 390 can be made of elastics; plastics, metals, and combinations thereof, however, other materials and geometries are within the scope of the invention as well. In one embodiment, rather than a headgear per se, other protraction devices are also contemplated to be within the scope of the invention, for example, protraction devices that could be anchored on other parts of the subject's body, or off a subject's body. In one embodiment, headgear 390 also enables application of extra-oral forces to a second face bow 306, which is represented only in part in FIG. 3 and is described and shown in more detail further below.

Referring to FIGS. 4a-c, there are seen top, front, side and perspective representations of an embodiment of a bone anchor. In one embodiment, a bone anchor 410 comprises a first end 403 configured to be coupled to extra-orally applied protraction forces, and a second end 404 structurally coupled to the first end. In one embodiment, bone anchor 410 comprises a connecting piece 420 that defines a length that couples the first end 403 to the second end 404 and that is configured to transfer forces from the first end to the second end. In one embodiment, the connecting piece comprises a linear portion 430 and a curved portion 440. In one embodiment the second end 404 comprises a plate 421 within which a plurality of apertures 422 are disposed. In one embodiment, apertures 422 are structured to receive bone screws of a type known in the dental surgery arts (for example, as represented in FIG. 14) to fixedly couple the plate 421 to an intra-oral location on the skull via an intra-oral tool adapted to facilitate installation of the screws. In one embodiment, the number of apertures is four apertures. As seen in the front view, in one embodiment, relative to a central axis defined by a portion of the connecting piece 420, two apertures 422 are disposed on one side of the axis and 2 apertures are disposed on another side of the axis. As seen in the side view, in one embodiment, relative to an axis defined by the length of the connecting piece 420, the plate 421 is disposed in a plane that passes along the axis. In one embodiment, plate 421 comprises a horizontal portion 423 from which two side portions 424 extend on either side. In one embodiment, two apertures 422 are disposed in the horizontal portion 423, and each side portion 424 is comprised of an aperture 422. In one embodiment, connecting piece 420 connects centrally to the horizontal portion 423 at a connecting portion 426 between each of the two side portions 424. In one embodiment, the connecting portion 426 is disposed centrally with respect to the horizontal portion 423 and equidistant from each of the apertures 422. In another embodiment, with a plate 421 comprising a horizontal portion having a vertical height at the central portion that is smaller or larger than shown in FIGS. 4a-c, the connecting portion 426 could be disposed either higher or lower such that the connecting portion would be disposed centrally on the horizontal portion but not equidistant from each of the apertures (see FIG. 4e). In one embodiment, the first end comprises a coupler 450. In one embodiment, coupler 450 is comprised of an aperture 451. In one embodiment, aperture 451 comprises a hollow cylinder. As seen in the front view in FIG. 4a, aperture 451 defines a central axis that is orthogonal to an axis defined by the connecting piece 420. In one embodiment, bone anchor 410 is manufactured from material having a hardness and or stiffness capable of allowing the shape of the bone anchor to be manually changed. In one example, the material comprises grade titanium and the exemplary dimensions shown in the figures, however, as will be seen from the description, other materials, geometries and dimensions are within the scope of the invention as long as they are compatible for human use and are sufficiently strong enough to transfer extra-oral protraction forces applied at the first end 403 to the second end 404. Although in an uninstalled configuration bone anchor 410 comprises the particular shape represented by FIGS. 4a-c, to better conform to the shape and geometry of a subject's intra-oral skeletal structure, in one embodiment, bone anchor 410 is manufactured to be capable of having its shape and geometry manually manipulated to fit a patient's bone geometry before installation by a clinician.

Referring to FIG. 5, there are seen top, a front, and perspective representations of an embodiment of a coupler. In one embodiment, a coupler 510 comprises a first end 560 configured to be coupled to extra-orally applied protraction forces and a second end 570 coupled to the first end 500. In one embodiment, coupler 510 comprises a body 562 configured to transfer forces from the first end to the second end. In one embodiment, the body 562 comprises an aperture 563 and a protrusion 564. In one embodiment, the aperture 563 is formed at the first end 560 and the protrusion 564 is formed at the second end 570. In one embodiment, the aperture 563 is configured to receive and to be coupled to a first end 203 of the first face bow 202 shown in FIG. 2. In one embodiment, the aperture 563 is configured to retain the first end of 203 such that with 200 to 1000 grams of force applied to each anchor via respective first ends 203 of the first face bow, couplers and ends of the face bow remain joined to each other. In one embodiment, second end 570 is configured to keep the first end 403 of the bone anchor 410 shown in FIG. 4 coupled to the second end 570 while an extra-oral force is applied to a second end 204 of the face bow 202 shown in FIG. 2. In one embodiment, protrusion 564 is configured to slideably and removeably fit within aperture 451 of the first end 403 of the bone anchor 410 shown in FIG. 4.

In another embodiment, the second end 570 of the coupler 510 can be configured to comprise an aperture at the location of the protrusion 564, and the first end 403 of the bone anchor 410 shown in FIG. 4 could be configured to comprise a protrusion at the location of the aperture 451. In one embodiment, coupler 510 is manufactured from nylon and with the exemplary dimensions shown in FIG. 5, however, other materials (for example, polymers/plastics), geometries and dimensions are within the scope of the invention as long as they are compatible for human use and are configured to transfer extra-oral forces to the bone anchor in the manner described above and further below.

With reference to FIG. 6, there is seen another embodiment of a first face bow. In one embodiment, the functionality provided by coupler 510 (see FIG. 5) is provided by an embodiment where the first ends of a first face bow 602 comprise an integral coupler 610. In one embodiment, first face bow 602 comprises ends 611 that are configured to couple to intra-orally installed bone anchor. In one embodiment ends 611 of first face bow 602 comprise a protrusion configured to fit within an appropriately dimensioned aperture of a first end of a bone anchor. In one embodiment, the ends 611 comprises a bent curved or hook like geometry formed at the same time as the formation of first face bow 602. Both the embodiment of FIG. 5 and FIG. 6 facilitate simple and quick connection and removal of a first face bow, not just by a dental specialist, but by a subject with the bone anchors installed.

With reference to the representations in FIGS. 7a-f, methods of use of a first face bow and bone anchor are described below.

In the discussion below, two exemplary examinations of different extra-oral forces as they are applied to a bone anchor via a first face bow are presented, where use of the terms forward horizontal, downward and vertical are used refer to a respective direction and orientation of a subject's skull when the subject is standing in a prone position with their head facing forward.

Unlike other devices, for example the Keles and De Clerck devices discussed in the background, which apply forces that cause rotation and thus unnatural downward movement and growth of the maxilla, the first face bow of the present invention is directed to treating maxillary deficiency and craniofacial dystrophy via a system, components and methods that, with reference to a standing subject's head facing forward, effectuate substantially only forward movement and growth of the maxilla. In one embodiment, the first face bow and bone anchors of the present invention are configured to apply forces to the maxilla that are uniquely able to generate positive forward growth not just of the maxilla, but as well as of the zygomatic bone and other bones that articulate with movements of the maxilla: sphenoid, frontal bone, ethmoid, etc.

The present invention identifies that when used with installed bone anchors, first face bow, and external headgear as seen in FIG. 4, the connecting portions 426 of each anchor is preferred to be maintained in the same plane (i.e. a horizontal plane when a patient is standing with their face pointing forward) the extra-oral second ends 204 the first face bow 302 is disposed in (see FIG. 3). Why this orientation is preferred is addressed in the examinations made below.

A first examination contemplates application of extra-oral protraction forces to a face bow 702 and couples the forces to a maxilla via a bone anchor 710 at an upward angle relative to a horizontal plane. With reference to FIG. 7a, the forces can be analyzed using the following equations, where along the Y Axis−F_{D} cos θ+F_{AY}=0, where F_{AY} is the Y component of F_{A}, where along the Z axis we get −F_{D} sin θ+F_{AZ}=0, and where the resulting moment will be M=F_{D} sin θ*L_{MY}+F_{D} COS θ*L_{MZ}. If F_{D} is approximately 500 g of force (4.9 N) acting at an angle of 30° and L_{MY}=5 cm and L_{MZ}=2 cm, we get the following result: F_{AY}=4.24 N=432 go force, F_{AZ}=2.45 N=250 g of force and M=0.25 Nm.

Using the above, we can estimate the load on screws that will be used to attach the bone anchor to the maxilla from the resulting moment by using the free body diagram shown in FIG. 7b. Assuming L_{S} is approximately 1 cm, we get screw shear forces in excess of the following. F_{S1}≈F_{S2}=25N=2500 g of force. This result reflects a balancing the moment M, which is primarily created by the Z component of F_{A}, combined with the large moment arm L_{MY}. The present invention identifies that a similar large moment could be generated by forces directed at a downward angle relative to a horizontal plane and, that at some value, such moment could be too large for the bone anchor screws and bone structure of the maxilla to handle.

In the second exemplary application represented by FIG. 7c, and to which an embodiment of the present invention is directed, extra-oral forces are applied by a face bow 702 to a maxilla horizontally. A similar analysis is performed as for the first application of forces above. With reference to FIG. 7c, along Y axis −F_{D}+F_{A}=0, where Z axis forces are all assumed to be zero, and the resulting moment is M=F_{D}*L_{MZ}. If F_{D} is approximately 500 g of force (4.9 N) and L_{MY}=5 cm L_{MZ}=1 cm, we get the following result: F_{D}=500 g of force and M=0.049 Nm. Thus, it is identified that in one embodiment, application of horizontal forces results in reduced moments being applied to the bone anchor screws and maxilla, which preferably reduces bone screw breakage and damage to the maxilla.

With reference to FIG. 7d, a frontal analysis of moment loads about the X axis is represented. In FIG. 7d, F_{A} appears as a force application point that points straight out from the figure. Depending on the application point, there will be a moment arm L_{MX} which will create additional moments on the bone anchor. To minimize this additional moment, application point of F_{A}, which corresponds to an end 204 of the face bow in FIG. 2, can be moved to align closer with a vertical plane in which the bone anchor is disposed. To further minimize stresses caused by the moment arm in FIG. 7d, the present invention identifies that a cross screw pattern of the apertures 422 provided on the bone anchor of FIG. 4 can be used minimize loading of the screws by providing equal distribution of forces that are transferred by the bone anchor to the connecting point 426.

Use of the present invention contemplates many embodiments. For example, another embodiment of the present invention is directed to application of extra-oral forces to the maxilla of a subject with a configuration that is intended to achieve optimal treatment of maxillary deficiency and craniofacial dystrophy. Further, one embodiment of the present invention is directed to application of extra-oral forces to the maxilla of a subject with a configuration that causes the maxilla and the bones that articulate with it to move and grow in a manner that achieves optimal treatment of maxillary deficiencies and craniofacial dystrophy. Also, one embodiment of the present invention is directed to application of extra-oral forces to an installed bone anchor with an orientation relative to the skull that causes no rotation or substantially no rotation at the bone anchor. Also, one embodiment of the present invention is directed to application of extra-oral forces to the maxilla of a subject with an orientation relative to the skull that causes no rotation or substantially no rotation of the maxilla about the bone anchor. Also, one embodiment of the present invention is directed to application of extra-oral forces to the maxilla with an orientation relative to the skull that causes no rotation or substantially no rotation of the bones that articulate with the maxilla about the bone anchor. In other embodiments, the present invention identifies that because rotational moments applied to bone anchor screws can be minimized, stresses applied to the bone anchor and bone anchors screws can minimized, where such minimization can be achieved when protraction forces applied to a face bow are applied at the face bow along a vector that passes through the plates of the bone anchors, for example in a forwardly directed upward direction as represented by FIG. 7e or a forward only direction as represented by FIG. 7f.

In embodiments, the zygomatic buttress of the maxilla and the infrazygomatic crest are identified by the present invention to be locations on the skull that are well suited for attaching bone anchors to achieve the benefits of the present invention, however, other attachment points are also within the scope of the invention, as long as the bone anchors and face bow are able to be dimensioned to allow application of forward or a combination of forward and upward vector extra-oral protraction vector forces in a manner described above and in a manner that interacts minimally with the lips and teeth of a particular subject. For example, in one embodiment it is contemplated that a bone anchor could be coupled higher on the skull along the zygomatic bone. However, it is identified that attachment to the zygomatic bone may require more invasive surgery, and as well, since the zygomatic bone articulates with significantly fewer bones than the maxilla, results achieved via attachment and application of forces to the zygomatic bone may potentially not be as beneficial to a subject as those that can be achieved via application of forces to the maxilla. Further discussions directed to the selection of locations for the attachment of bone anchors is provided below.

Referring to FIGS. 8a-d, there are seen representations of top, front, side, and perspective and views of an embodiment of the second face bow shown partially in FIG. 3 and more fully in FIG. 1. In one embodiment, a second face bow 802 comprises first ends 803 configured to be inserted into the oral cavity of a subject and second 804 and third ends 805 configured to be coupled extra-orally to a headgear or other type of protraction device worn by a subject (see FIG. 3, which represents a coupling of third ends of a second face bow to a headgear). FIGS. 8a-d show the second ends 804 and 805 being comprised of a straight piece and a loop respectively, however, other geometries for the second ends are considered to be within the scope of the invention as long as such geometries enable the ends to be coupled to an extra-oral headgear or other external structure capable of applying extra-oral forces to the ends. In one embodiment the first face bow 902 comprises 304V stainless steel and the exemplary dimensions shown in FIGS. 8a-d, however, other materials, geometries and dimensions are within the scope of the invention as long as they are compatible for human use and are configured to transfer extra-oral forces applied at the second 804 and third ends 805 to the first ends 803 in the manner described above and further below.

Referring to FIGS. 9a-b, there are seen representations of top and side views of another embodiment a second face bow. As in the embodiment of FIGS. 8a-d, a second face bow 902 comprises first ends 903 configured to be inserted into the oral cavity of a subject and second 904 and third ends 905 configured to be coupled extra-orally to a headgear worn by a subject. In embodiments, ends 904 and 905 comprises hooks or loops. In one embodiment, the first ends 903 are integrated with a material comprised of silicone, or other bio-compatible polymer, plastic or acrylic. In one embodiment, the material is configured to fit against each side and against the maxilla behind the molars and provide a cushioning fit of the first ends 903 against the maxilla (i.e. see FIG. 1 for the location where intra-oral first ends of a second face bow are disposed). In one embodiment, the locations where first ends 903 are coupled to the maxilla at maxillary tuberosities. In one embodiment, second face bow 902 is configured to receive extra-oral forces at third ends 905 and transfer the forces intra-orally to the maxilla via the first ends 903. In one embodiment, horizontal or substantially horizontal extra-oral forces are applied to second face bow 902 with respect to the skull of a prone subject such that when inserted and coupled to the maxilla, a first end 903 of the second face bow 902 and the point where extra-oral forces are applied at a third end 905 are aligned in the same horizontal plane (i.e. see orientation of second face bow 902 in FIG. 3). In one embodiment, extra-oral forces are applied to the second face bow 902 by a headgear. Application of extra-oral forces horizontally to the maxilla via a second face bow 902 can be used to achieve the same benefits as that described above with use of the first face bow but without the intra-oral surgery needed to attach a bone anchor to the maxilla. In an alternative embodiment (see FIG. 9c), it is contemplated that intra-oral ends of a second face bow as discussed above can be embedded within an acrylic appliance that is configured to fit against the maxilla at superior palate either behind the molars or the maxillary tuberosity.

Referring to FIG. 10, there is seen a representation of application of horizontal and upward forces by a second face bow to a maxilla of a subject. The present invention identifies that in some individuals, in order to achieve the best overall maxillary and facial bone growth, in addition to the use of horizontal or substantially horizontal forward force vectors to achieve forward growth of the maxilla and skeletal bones coupled to the maxilla, varying degrees of upward force may also be desired to be applied. Although application of upward force could be achieved via a configuration of the first face bow where its extra-oral end is positioned in a higher horizontal plane than the bone anchor, as identified above, such an orientation can cause moments to be generated that can cause undesired excessive stresses to be applied to bone anchor screws and thus the maxilla. Reduction of this stress is discussed below.

In one embodiment, a headgear 1090, elastics 1091, first 1003, second 1004, and third 1005 ends of a second face bow 1002 are used to apply both forward forces, as well as forwardly directed upward forces to a subject. With reference to the representations of the first and second face bows in FIG. 3, when horizontal or substantially horizontal forces are applied directly to bone anchors by a first face bow alone, it is identified that the forces need to fight against the resistance of all the bones coupled to, and that articulate with, the maxilla. It is identified that additional application of forward forces to the maxilla by a second face bow (see horizontal orientation of elastic 1091 connected to third end 1005) enables the amount of force applied by the first face bow to be reduced, which can in turn be used to reduce the amount of stress applied to bone screws and the maxilla. The present invention also identifies that although it may not be desired to apply upwardly directed force vectors to the bone anchor, and thus the maxilla, by the first face bow, such forces could also be applied by the second face bow at 1004 (see upward angle of elastic 1091 connected to second end 1004) without causing excessive stresses on bone screws, because the second face bow does not require use of bone screws. Thus it will be appreciated by those skilled in the orthodontic arts that treatment of maxillary deficiency and craniofacial dystrophy by the present invention encompasses not just horizontal or substantially horizontal application of forces to the maxilla, but as needed, varying degrees of upwardly directed forward forces as may be needed to mimic both the beneficial forward as well as upward force production that the tongue applies intra-orally to the structure of the mouth.

The bone anchor described herein is an innovative orthodontic anchor designed to be used to provide orthodontic maxillary protraction as well as in other orthodontic procedures that require orthodontic anchorage: (molar distalization, for example, or mandibular forward positioning). The device's design is innovative in that it allows optimization of force distributions as well as force vectors.

The present invention further identifies that recent innovations in additive manufacturing can be used to create customized bone anchors according to information obtained during software analysis of patient specific 3D data/model. Such a customized approach has several advantages. Namely, it eliminates an installing surgeon from being having to manually bend bone anchors to fit to a patient's skeletal structure prior to intra-oral installation, where such manipulation can degrade mechanical properties of the bone anchor and subject it to fracture or malperformance, as well as entail uncertainty and time consumption during installation. A consequence of not needing to manually manipulate the bone anchor shape manually is that stiffer materials than otherwise could be used can be considered, which opens up the possibility of other applications for the present invention.

Referring to FIGS. 15a-b, there are seen representations of embodiments of a bone anchor. The bone anchor embodiments of FIGS. 15a-b have in common with other embodiments described previously in that they comprise: a top second end 1504 configured to couple extra-oral forces to a patient's maxilla, where the forces are first received at a bottom first end 1503, which is coupled to the top second end 1504 via a connecting piece 1520 that defines a distance that separates the bottom first end and the top second end.

In the representations of FIGS. 15a-b, the top second end 1504 of each bone anchor 1510 is configured to be attached to a zygomatic buttress of the maxilla, however, it is identified that such attachment may not necessarily be against a completely flat surface. Thus, although the top second end was previously represented with an initially flat surface (see FIG. 4 above), to achieve good fitment against the zygomatic buttress, in one embodiment, the top second end 1504 is configured with other than a completely flat surface, for example, in the form of a plate 1521 comprised of contoured surface configured to match a surface of a desired point of attachment against the maxilla.

Referring to FIG. 16, there is seen another representation of a bone anchor. In one embodiment, reduction in interference with a patient's normal oral functions by a bone anchor 1610 can be achieved by customizing the length and/or geometry of a connecting piece 1620 to comprise one or more of straight, bent, curved, and/or twisted portion. In embodiments, connecting piece 1620 comprises the same or a varying cross-sectional geometry along one or more portions of its length. In embodiments, a surface of bone anchor 1610 at a cross-section taken through bone anchor 1610 comprises one or more of a generally round, elliptical, semicircular, curved and flat side. For example, in one embodiment, a top portion 1620a of connecting piece 1620 comprises a surface comprised of cross-sections having areas defined by at least one flat or curved side and a rounded side, a middle portion 1620b that comprises a twisted and curved surface comprised of smaller cross-sectional areas than at the top portion and that are defined by at least one flat side or curved side and a rounded side, and a bottom portion 1620c that includes a curved surface comprised of cross-sectional areas that are defined by one or more rounded side.

Referring to FIG. 17, there are seen virtual representations of a bone anchor. In one embodiment, the present invention comprise one or more module embodied as instructions stored on or in a computer readable medium in the form of software, hardware, or firmware and that are interpreted by a processor to enable creation, manipulation, and display of a 3D representation of a bone anchor 1710a on a user interface or display. In one embodiment, the one or more module is used to import or generate a first stereolithography (STL) file representative of the 3D representation 1710a. In one embodiment, one or more module is configured to allow a user to manipulate the shape and orientation of the 3D representation 1710a to fit against the surface of the 3d representation of a patient's maxilla. In one embodiment, after manipulation, the processor is configured to save a manipulated version 1710b of the 3D representation 1710a as a second STL file. In one embodiment, a printer is configured to use the second STL file to create a physical copy of the manipulated version 1710b.

Referring to FIG. 18, there is seen a representation of the location of a zygomatic maxillary suture. As known to those skilled in the art, human skulls comprise a plurality of anatomic sections that are joined by sutures. In FIG. 18, line “L” points to a typical location of a zygomatic maxillary suture that separate maxillary (left of the suture) and zygomatic (right of the suture) portions of a skull.

Referring to FIG. 19, there is seen a representation of a human skull. In FIG. 19, dots with different shades indicate variability of the maxillary cortical bone thickness at a different locations on a skull, where the darkest dots indicate more thickness than lighter dots, where each dot is separated by about 5 mm. In FIG. 19, a virtual representation of a bone anchor 1910 that has not been fully manipulated is overlayed over the representation of the skull to illustrate how screw holes in its plate 1921 are desired to be configured to overlay thicker regions (darker dots) of the zygomatic buttress of the maxilla. In FIG. 19, plate 1921 comprises a right/distal portion that is generally slanted with respect to a central axis defined by a top portion of connecting piece 1920 and so as to generally match the slope/slant of the zygomaticomaxillary suture of the skull and so as to take advantage of the thick portions of the skull bone adjacent the suture. In one embodiment, the plate also comprises an left/medial portion that is generally in alignment with the axis, so that when coupled to the maxillary buttress, screws used to attach the bone anchor will be better positioned to avoid thinner portions of bone that is typically present in the anterior sinus region of the skull.

As discussed above, the present invention enables the shape of bone anchors to be customized to the structure and shape of each patient's skull. To achieve such customization, in one embodiment, the present invention utilizes software analysis of patient specific 3D data derived from CBCT, CT, or MM scan. After processing patient image data, each patient's skull and bone thickness and geometry is derived from the data to create a model of the skull that can be displayed in 3D. The 3D model and data can be used by a technician or clinician to design an optimized bone anchor by manipulating an initial virtual representation of a bone anchor created from a first STL file (see 1710a in FIG. 17) so that it fits to and along contours of the 3D model at locations that are optimized for mounting screws. During manipulation of data, geometrical changes to the bone anchor can be visualized in real-time, for example, a shape, length and/or thickness of a connecting piece can increased or decreased so that it properly exits keratinized tissue, or at another desired location, where the plate is intended to be mounted, while at the same time taking into account a desired location for where a bottom first end of the bone anchor is desired to be positioned. Further, during manipulation, a second top end of the bone anchor may many be manipulated so that the surface of its plate conforms to the surface of the patient's skull at a desired point of attachment.

In one embodiment, virtual manipulation of a bone anchor includes placement of it's second top end along the posterior/superior portion of a patient's maxilla while positioning it's first bottom end roughly 2 mm above their gum line, where in actual use, this location typically provides thicker bone structure for mounting of bone screws, and a mounting point that is close to the center of resistance of the maxilla and that is close to the zygomaticomaxillary suture. In one embodiment, once the location of the suture has been established, a goal is to place a distal edge of the bone anchor plate along the suture line while having the neck drop between the first and second molar. It is identified, however, that for some patient's having bone thickness determined to be different from the representative skull of FIG. 19, optimal location of a first end and second end of a bone anchor may be different from that described above.

After manipulating a virtual bone anchor to obtain a desired fit to a particular patient's geometry and/or to the thick portions of the maxilla, data representative of it manipulated shape is saved as a second STL file, which data can subsequently be used to manufacture a physical bone anchor. In one embodiment, bone anchors according to the present invention are manufactured via additive 3D printing by using data stored in the second STL file. In one embodiment, to facilitate easier manipulation of a virtual representation of a top second end and connecting piece of a bone anchor, the first STL file comprises separate data representative of a virtual bone anchor that does not include a bottom first end, and separate data representative of a virtual bottom first end. In one embodiment, after the virtual bone anchor sans a bottom first end is manipulated to fit a particular location on the skull (see 1710b in FIG. 17), the virtual representation of the bottom first end is displayed, and the bottom first end with a desired shape and in a desired orientation is virtually joined aligned to a bottom portion of the connecting piece. After joining and alignment, data representative of a complete bone anchor is saved in a second file, and the second file can be used to manufacture the bone anchor, for example, as represented by bone anchor 2010 in FIG. 20. In one embodiment, the second file comprise an STL file that is used to manufacture the bone anchor via 3D printing in metal as is known to those skilled in the art. In one embodiment, printing is performed using grade 5 titanium or other material having a similar hardness and/or stiffness. During virtual manipulation, in some embodiments, the relationship of the geometry of one part of a bone anchor may be manipulated relative to another part to achieve optimal strength for a particular amount of force desired to be applied to the bone anchor. For example, in one embodiment, the following equation may apply: (neck width)=(neck length)/10, however. However, this relationship can vary based on different applications and different material uses, for example, stiffer materials may require less thickness.

Referring to FIG. 21, there is seen a representation of the anatomy of a skull. Although coupling of a bone anchor has been described as being preferably to certain regions of a patient's maxilla along a maxillary buttress (i.e. zygomaticomaxillary buttress), it is understood that depending on a particular patient's skeletal geometry or a particular other application, bone anchors of the present invention can be configured to be coupled to fit against other portions of a patient's maxilla, including, but not limited to the nasomaxillary buttress or to the nasomaxillary buttress and the zygomaticomaxillary buttress.

Referring to FIGS. 22a-b, there are seen embodiments of bone anchors that are customized to provide optimized anchorage given a patient's specific geometry and anatomy, as well as desired end use/orthodontic procedure. Referring back to FIGS. 4a-c, there is seen a representation of a bone anchor which comprises a generally flat plate at its top second end, a connecting piece with a middle portion that is generally straight and rounded in a cross-section, and a bottom first end that comprises a cylindrical connector having an aperture defined by an axis that is aligned generally parallel to plane in which the flat plane is disposed. In FIGS. 4a-c, the plate comprises two sides that generally symmetrical with respect to each other about an axis defined by the connecting piece, where each side of the plate comprises two apertures and where a center location of the apertures can be generally defined by corners of a square. As also described above, the shape and geometry of a bone anchor can differ from that represented by FIGS. 4a-c. For example, as represented by FIG. 20 a plate of a comprises a left and right portion, wherein the left and right portion each comprise at least two apertures, wherein apertures in the right portion are disposed along an axis that is generally slanted with respect to an axis of the connecting piece at its connection to the plate, and wherein the apertures in the left portion are disposed along an axis that is generally parallel with respect to the axis of the connecting piece. In the embodiment of FIG. 20, the center of the apertures can be defined by corners of a polygon that has two non-parallel sides, where the center of each of the two of the apertures on either side of the connecting piece correspond to the ends of the non-parallel sides. It is understood that the geometry represented by FIG. 20, as well as other geometries within the scope of the invention, may be reversed for a bone anchor used on an opposite side of the skull. In the embodiment of FIG. 20, an orientation of an axis of a cylindrical connector at the first bottom end is disposed in an orientation that is generally orthogonal to a plane a plate at the second top end is generally disposed in. As represented in FIGS. 22a-b, in embodiment, an orientation of an axis of a cylindrical connector at the first bottom end is disposed in an orientation that is generally parallel to a plane the plate at the second top, however in FIG. 22b, apertures in a plate at the top second end are disposed in other than at the corners of a square or polygon. In one embodiment, the plate is asymmetrically disposed about an axis defined by a top portion of a connecting piece. In one embodiment, apertures in the plate at the top second end of a bone anchor are disposed generally in a linear relationship of three apertures. In one embodiment, apertures in the plate define an L-shaped relationship of 4 apertures. In other embodiments, depending on the amount of available intra-oral geometry or a desired amount of force needed to secure a bone anchor to a particular portion of the skull, a bone anchor can be manufactured to comprise more or fewer apertures in its plate

Those skilled in the art will identify that many other embodiments are also within the scope of the present invention, which should be limited only by the extent of the present or future claims presented along with the present application.

What is claimed is:

1. (canceled)

2. The system of claim 3, wherein the face bow consists of the one or more coupler.

3. A system to treat maxillary deficiency, the system comprising:
an orthodontic face bow comprising an intra-oral portion; and
an extra-oral portion,
wherein the extra-oral portion is configured to receive one or more extra-oral protraction force,
the intra-oral portion comprises one or more coupler configured to transfer the one or more extra-oral protraction force to intra-oral portions of the patient's mouth that are not teeth of the patient, and
the one or more coupler comprises a bone anchor.

4. The system of claim 3, wherein the intra-oral portions of the patient's mouth that are not teeth comprise maxilla of the patient.

5. The system of claim 3, wherein the intra-oral end comprises silicone, plastic, acrylic, polymer, or combination thereof.

6. The system of claim 5, wherein the intra-oral portions of the patient's mouth that are not teeth comprise a maxilla.

7. The system of claim 3, further comprising one or more protraction device configured to apply the one or more extra-oral force.

8-102. (canceled)
